SEQUENCE LISTING (28 sequences)

SEQ ID NO: 1 - DNA, 2418 bp, Artificial
  Medicago truncatula
atggctgcttgtgctttgttcaatggtgctactactgatgaaaccaacaacaatagtgcttcaaactccagcaatatcagtagtgaagatttacacactctacacaacagcaacaccaacatgccacaacaacaacaacctcattctgatagaaaagtgcttagaaaaagaatggcttctgaaatggaacttcaacttcaagtccacaacaacaacaatagattttctcgtagaaacaacaacaacatttcctcattgaattgttctttaccttcttccacagaaaaaggtgttacaacaacaacaacaacaacactagccgcaggtacaaataataatgctaataataataactttcattataatactaataatataaacaatagtagtcatcatagtaatgttgctttgtcacgagataacgttgctgttcaaaattaccccactgtgaccgtcacaaccaactactccactatgttgctaccttcttcttccaacaatctcaacaattcctcaacctcaaactacgctcattttcaacaaccacttgttgaggaacaaaaccatgttcctaatatttgtggtttttctggtttaccactttttccatctcaaaaccaacccaacagaaccagcaacagaaacagtaacagtaacataagttctgctactaatattgttgatgttgttaactcttctacaccttctatgatggacgaaacatcagctacaacaaactggattgatggtattttgaaggatcttattcatacttcaaacagtgtttctattcctcaacttatcaacaatgttagagagattatatatccttgtaatccaaaccttgctcttgttcttgaacataggttacgtcttcttacagaaactgctccaagtgttgttcctgagagaaaaagaaacaacactgaacaacaaagtgtttctaatgttaatgttttacctgcttctaatgttaattcctcagtcaaactcatgaaccgtgttgatgacattgttccacatttttctgattcatcaactcttttgaatcaaaatcaaaacatgtttcctaattggggtgtgccacaaatcaataacaacaataaccctttggttactttaccttcacaaccacaatcaacacaacaagatcaacatcaacatcaacaacatcaagagcatcaagaagatcttgtacctgcaacaacaccaccaccaccaacttctgctgagttagcaataacaagaaaaaagaaagaggagattaaagaacagaagaagaaagatgaagaaggtttacaccttctaacattacttcttcaatgtgcagaagcagtatcagcagagaatcttgaacaagcaaataagatgttactcgaaatttcgcagctttctacgcctttcgggacctcggcacaaagagtagcagcttatttctcagaagcaatttcagcaagactagtgagttcatgtttaggaatctatgcaacacttccaccacacacactacataatcaaaaagttgcttcagcttttcaagtcttcaatggtattagtccatttgtgaaattctctcatttcactgctaaccaagctatacaagaagcttttgatagagaagaaagagttcacatcatagatcttgatatcatgcaaggtttacaatggcctggtctttttcatatccttgcttcaagaccaggtggacctccttatgtgagactcactggattaggtacttccatggaaacccttgaagctactggtaaacgtttgtctgattttgcatctaaacttggtcttccttttgagttctttcctgttgctgagaaagttggaaatattgatgttgagaagcttaatgttagtaaatcagaagctgttgctgttcattggttgcaacattctctatatgatgttactggttcagatacaaacactttatggctcttgcaaaggttggcacctaaagtggtaacagtagtggaacaagacttgagcaatgcaggctcattcttgggaaggtttgtggaagcaatacattactactcagcattatttgattctcttggttcaagctatggagaagagagtgaagagagacatgtggtggagcagcaactattatcaagagagattcgtaacgtacttgcagttggtggaccatcaaggagtggtgagataaagtttcataattggagagaaaagcttcaacagtgtggttttagaggtatctctttggctggtaatgcagctacacaagctagtcttcttcttggcatgttcccttctgaagggtatactttggttgaggataatggtatccttaaacttggttggaaagatctttgtttgcttactgcttctgcttggagacctccctttcataccaacaatatcataccacatcataactga

SEQ ID NO: 2 - DNA, 1476 bp, Artificial
  Medicago truncatula
atggatacattgtttagacttgtcagttttcaaccacaccaacaacaagaagatcaatctctcaactccaccaccagcagaacaacaacatcaagtagctctcgatcctcaaaacaaaactatcaccaatattacaatccacaagaagttgaagaagaatgcttcaactttttcatggatgaagaagacctttcctcatcttcttccaaacactactatccttataatcaacctcaaacctcaaatactattactacaaacacccccacaacatcactcacaccccctccaccaacagctgatttcaccttcgaattaaccggaaggtgggccaacaacatcctcctcgaaacagcacgagccatagcagaaaaaaacagcactcgtttacaacaactcatgtggatgcttaacgaactaagctccccttatggtgacatagaacaaaaactagctgcatattttctccaagccttattctctcgaatgaccgaagccggtacccgaaccttccgaaccctagcttcagcatcagaaaaaacttgctctttcgaatcaacaagaaaaacggttctaaaattccaagaagttagtccgtggacaaccttcggccatgtcgcatgcaatggcgccattttagaagccttcgaaggtgattccaagctgcacataattgatattagtaacacttattgcactcagtggccaactctttttgaagcgttggccactcgtgctgacgatactcctcacctccgtctaactaccatcgtcactgctggtggttctgttcaaaaagttatgaaggaaattggtgctagaatggaaaaattcgctagactcatgggagttccatttaaattcaacgttattcatcactccggcgatctttctgatttgaattttttggatttagatattaaagaagatgaagctttagctgttaattgtgttaatgctttgcattcagtaacagttggcaatggtaatggtaacggtaataaccgtcgtgactcgttgatagcttcgttgatcgcgttgcggcctaggatagtaacaatggttgaagaggaggctgatttaaattttggtaatgaaggatatgaatttgtgaatggttttgaagagtgtttaaggtggtttagggtttactttgaagcattagaagagagttttccgaagacgagtaacgagcgtttgatgcttgaaagggaagcagggagagggatagttgatttggtggcatgtgctccggcggagtcgattgaacggagagaaactgcggtgaggtggtcacggcgtttgcatggtcgagggtttaatacggtggcgtttagtgaggaagtttgtgatgatgttagggctttgttgaggaggtataaggaagggtggtcgatgatacggtgtaatgatgccggaatattcttgacgtggaaggaacaaccggtggtgtgggccagtgcatggaggccttga

SEQ ID NO: 3 - protein, 805 aa, Artificial
  Medicago truncatula
MAACALFNGATTDETNNNSASNSSNISSEDLHTLHNSNTNMPQQQQPHSDRKVLRKRMASEMELQLQVHNNNNRFSRRNNNNISSLNCSLPSSTEKGVTTTTTTTLAAGTNNNANNNNFHYNTNNINNSSHHSNVALSRDNVAVQNYPTVTVTTNYSTMLLPSSSNNLNNSSTSNYAHFQQPLVEEQNHVPNICGFSGLPLFPSQNQPNRTSNRNSNSNISSATNIVDVVNSSTPSMMDETSATTNWIDGILKDLIHTSNSVSIPQLINNVREIIYPCNPNLALVLEHRLRLLTETAPSVVPERKRNNTEQQSVSNVNVLPASNVNSSVKLMNRVDDIVPHFSDSSTLLNQNQNMFPNWGVPQINNNNNPLVTLPSQPQSTQQDQHQHQQHQEHQEDLVPATTPPPPTSAELAITRKKKEEIKEQKKKDEEGLHLLTLLLQCAEAVSAENLEQANKMLLEISQLSTPFGTSAQRVAAYFSEAISARLVSSCLGIYATLPPHTLHNQKVASAFQVFNGISPFVKFSHFTANQAIQEAFDREERVHIIDLDIMQGLQWPGLFHILASRPGGPPYVRLTGLGTSMETLEATGKRLSDFASKLGLPFEFFPVAEKVGNIDVEKLNVSKSEAVAVHWLQHSLYDVTGSDTNTLWLLQRLAPKVVTVVEQDLSNAGSFLGRFVEAIHYYSALFDSLGSSYGEESEERHVVEQQLLSREIRNVLAVGGPSRSGEIKFHNWREKLQQCGFRGISLAGNAATQASLLLGMFPSEGYTLVEDNGILKLGWKDLCLLTASAWRPPFHTNNIIPHHN

SEQ ID NO: 4 - protein, 491 aa, Artificial
  Medicago truncatula
MDTLFRLVSFQPHQQQEDQSLNSTTSRTTTSSSSRSSKQNYHQYYNPQEVEEECFNFFMDEEDLSSSSSKHYYPYNQPQTSNTITTNTPTTSLTPPPPTADFTFELTGRWANNILLETARAIAEKNSTRLQQLMWMLNELSSPYGDIEQKLAAYFLQALFSRMTEAGTRTFRTLASASEKTCSFESTRKTVLKFQEVSPWTTFGHVACNGAILEAFEGDSKLHIIDISNTYCTQWPTLFEALATRADDTPHLRLTTIVTAGGSVQKVMKEIGARMEKFARLMGVPFKFNVIHHSGDLSDLNFLDLDIKEDEALAVNCVNALHSVTVGNGNGNGNNRRDSLIASLIALRPRIVTMVEEEADLNFGNEGYEFVNGFEECLRWFRVYFEALEESFPKTSNERLMLEREAGRGIVDLVACAPAESIERRETAVRWSRRLHGRGFNTVAFSEEVCDDVRALLRRYKEGWSMIRCNDAGIFLTWKEQPVVWASAWRP

SEQ ID NO: 5-14 - skipped (10 entries)

SEQ ID NO: 15 - DNA, 12 bp, Artificial
  synthetic
aaaaaaaatatt

SEQ ID NO: 16 - DNA, 12 bp, Artificial
  synthetic
aatattttttat

SEQ ID NO: 17 - DNA, 12 bp, Artificial
  synthetic
aattttttttta

SEQ ID NO: 18 - DNA, 12 bp, Artificial
  synthetic
aatatttttttt

SEQ ID NO: 19 - DNA, 12 bp, Artificial
  synthetic
aatatatttttt

SEQ ID NO: 20 - DNA, 12 bp, Artificial
  synthetic
gatacaaatatt

SEQ ID NO: 21 - DNA, 11 bp, Artificial
  synthetic
aatatttattt

SEQ ID NO: 22 - DNA, 12 bp, Artificial
  synthetic
aatattttattt

SEQ ID NO: 23 - DNA, 12 bp, Artificial
  synthetic
aacgaaaatatt

SEQ ID NO: 24 - DNA, 12 bp, Artificial
  synthetic
aatatttttttt

SEQ ID NO: 25 - DNA, 31 bp, Artificial
  synthetic
cgggatcctatggatacattgtttagacttg

SEQ ID NO: 26 - DNA, 31 bp, Artificial
  synthetic
ccggaattcctcaaggcctccatgcactggc

SEQ ID NO: 27 - DNA, 39 bp, Artificial
  synthetic
ctagatctggatctagaactccgtttgggtttcgcttaa

SEQ ID NO: 28 - DNA, 12 bp, Artificial
  synthetic
aatatttttatt

(19) United States
(12) Patent Application Publication
(10) Pub. No.: US 2024/0287624 A1
(43) Pub. Date: Aug. 29, 2024

(54) Use of SHR-SCR in Leguminous Cortical Cell Fate Determination and Non-leguminous Cortical Cell Division Potential Modification

(71) Applicant: CAS Center for Excellence in Molecular Plant Sciences, Shanghai (CN)

(72) Inventors: Ertao Wang, Shanghai (CN); Wentao Dong, Shanghai (CN); Yayun Zhu, Shanghai (CN); Huizhong Chang, Shanghai (CN)

(21) Appl. No.: 18/001,541

(22) PCT Filed: May 19, 2021
(86) PCT No.: PCT/CN2021/094574

§ 371 Date: Jul. 11, 2023

(30) Foreign Application Priority Data
Jun. 15, 2020 (CN) 202010543164.4

(51) Int. Cl. C12Q 1/6895; A01H 1/04

(52) U.S. Cl. CPC C12Q 1/6895; A01H 1/04

(57) ABSTRACT

The invention belongs to the technical field of biotechnology and botany, relating to a method for modifying cell identity and regulating root nodule symbiosis, and specifically relating to the use of a new in-vivo plant mechanism formed on the basis of SHR-SCR in modifying the division potential of cortical cells. Also provided is a novel approach for identifying the traits of plants, thereby providing a feasible method for plant identification and an effective tool for plant breeding and screening.

### RELATED APPLICATIONS

This application is a National Stage Application under 35 U.S.C. 371 of expired PCT application PCT/CN2021/094574 designating the United States and filed May 19, 2021; which claims the benefit of CN application number 202010543164.4 and filed Jun. 15, 2020, each of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Jul. 7, 2023, is named “008703.00031_ST25.txt” and is 19726 kb in size.

### TECHNICAL FIELD

The disclosure belongs to the technical field of biotechnology and botany. More specifically, the disclosure relates to a method for modifying cells and regulating rhizobial symbiosis.

### TECHNICAL BACKGROUND

Symbiotic nitrogen fixation is a mutually beneficial symbiosis between plants and nitrogen-fixing microorganisms. According to the difference of symbiotic bacteria, symbiosis bacteria can be divided into three types: Cyanobacteria, Actinorhiza or Rhizobia. Usually, the so-called rhizobial symbiosis is the symbiosis between leguminous plants and rhizobia. Root nodule, a new organ formed in plants by rhizobia, converts free nitrogen in the air into nitrogen-containing compounds for plant growth and enhances the ability of legumes to adapt to low-nitrogen fertilizer soils. At the same time, legumes provide suitable environment and growth-necessary carbohydrates for rhizobia. In addition, after legumes died, fixed nitrogen will be released to soils and utilized by other plants, fertilizing the soil. Rhizobial symbiosis is vital to maintain the cycle of nitrogen on earth and nitrogen metabolism of plants.

At present, China's agricultural cultivation mainly depends on nitrogen fertilizer from outside. Over-fertilization of nitrogen has caused serious environmental problems such as surface pollution, groundwater contamination and soil acidification, which is one of important reasons for destroying ecological balance and a serious threat to sustainable agricultural development. How to solve these problems? Besides rational application of chemical fertilizer, a more important way is to achieve nodulation of non-leguminous plants through the study of rhizobial symbiosis.

In recent years, with the development of a series of subjects such as molecular biology and bioinformatics, a signal pathway for the symbiosis of legumes and rhizobia has been basically established. However, regulatory mechanisms of cell division are still poorly understood. In the process of establishing symbiosis between plants and symbiotic bacteria, cortical-dividing cells of host plants have an ability to accommodate nitrogen-fixing bacteria and can carry out symbiotic nitrogen fixation. Interestingly, nitrogen-fixing bacteria cannot invade dividing lateral root primordia cells. For legumes, the nodule is mainly derived from cortical cell division. Therefore, the division potential of cortical cells is a prerequisite for the establishment of symbiosis. So, an in-depth study of genetic basis of root cortical cell division potential of legumes not only contributes to clarifying molecular mechanisms of rhizobial symbiosis, but also lays a theoretical foundation for symbiotic nitrogen fixation in non-legumes.

Therefore, in this field, it is necessary to research in depth on cell division regulation mechanisms of rhizobial symbiosis in order to clarify mechanisms of the nodule formation in legumes and improve symbiotic possibilities by using genetic engineering technology to modify non-legumes.

### SUMMARY OF THE DISCLOSURE

The purpose of the disclosure is to provide a method for modifying the division potential of root cortical cells in plants and finally to induce the nodulation of non-legumes.

In the first aspect, the present disclosure provides a method for identifying plant traits, comprising: analyzing the promoter of plants' gene SCARECROW; wherein, if cis-elements AT1 Box and Enhancer are both present, it indicates that the gene SCARECROW is expressed normally and the plant traits are normal; if either AT1 Box or Enhancer is absent, it indicates that the gene SCARECROW is expressed abnormally and the plant traits are abnormal; wherein, the traits comprise formation of infection threads, abilities of cortical cells to respond to cytokinins and rhizobial infection, NIN-mediated plant spontaneous nodulation, cortical cell division or nodule organogenesis.

In another aspect, the present disclosure provides a method for selecting plants with normal traits, comprising: analyzing the promoter of plants' gene SCARECROW; wherein, if cis-elements AT1 Box and Enhancer are both present, it indicates that the gene SCARECROW is expressed normally and the plant traits are normal; wherein, the traits comprise formation of infection threads, abilities of cortical cells to respond to cytokinins and rhizobial infection, NIN-mediated plant spontaneous nodulation, cortical cell division or nodule organogenesis.

In another aspect, the present disclosure provides a use of promoter of gene SCARECROW, for identifying plant traits; or, for directionally-screening plants with normal traits; wherein the traits comprise: formation of infection threads, abilities of cortical cells to respond to cytokinins and rhizobial infection, NIN-mediated plant spontaneous nodulation, cortical cell division or nodule organogenesis; preferably, the identification is based on presence of cis-elements AT1 Box and Enhancer in the promoter of gene SCARECROW.

In a preferred embodiment, the presence of both AT1 Box and Enhancer indicates that cortical cell division or cortical biomass is normal; the absence of either indicates that cortical cell division or cortical biomass is abnormal.

In a preferred embodiment, the plant with normal traits is a plant that forms root nodules or nodule-like tissues (for example, nodule-like protrusions).

In a preferred embodiment, the cis-element AT1 Box has a nucleotide sequence shown in SEQ ID NO: 28 or a nucleotide sequence with more than 80% (such as more than 83%, 85%, 90% or 95%) sequence identity to the nucleotide sequence shown in SEQ ID NO: 28; preferably, it comprises a nucleotide sequence selected from any one of SEQ ID NO: 15-24.

The sequence of cis-element Enhancer is GANTTNC, wherein the N represents A, T, C or G; preferably, it has a nucleotide sequence shown in any one of SEQ ID NO: 5-14.

In a preferred embodiment, the plant is selected from the following groups comprising: plants expressing gene SCARECROW; rhizobial plants; preferably comprise Leguminosae (legumes); more preferably, comprise (but are not limited to) Medicago truncatula, Glycine, Lotus, Pisum, Cicer, Lupinus, Phaseolus, Trifolium and Parasponia; Gramineae; preferably comprise (but are not limited to): rice, barley, wheat, oats, rye, corn, sorghum; and/or Brassicaceae.

In another aspect, the present disclosure provides a method for improving traits of legumes or Gramineous plants, comprising improve the expression or activity of SCARECROW and SHORT ROOT in plants, or promote the interaction of SCARECROW and SHORTROOT; wherein the improved traits are selected from the following group comprising: promoting the formation of infection threads, changing identity of cortical cells, improving abilities of cortical cells to respond to cytokinins, improving ability of cortical cells to respond to rhizobial infection, promoting NIN-mediated plant spontaneous nodulation, promoting cortical cell division, promoting nodule organogenesis.

In a preferred embodiment, the promotion or improvement represents significant promotion or improvement, such as promotion or improvement by 20%, 40%, 60%, 80%, 90% or higher.

In a preferred embodiment, the promotion of root nodule formation is the formation of root nodules or root nodule-like tissues without inoculation of rhizobia.

In a preferred embodiment, SCARECROW and/or SHORT ROOT are ectopically expressed in the cortex (that is, it is located and expressed in the cortex); preferably, the ectopic expression is ectopic over-expression.

In a preferred embodiment, the expression is performed using a cortical cell-specific promoter or a ubiquitous promoter.

In a preferred embodiment, the cortical cell-specific promoter comprises: NRT1.3 promoter (pNRT1.3).

In a preferred embodiment, the ubiquitous promoter comprises: LjUBQ promoter (pLjUBQ).

In a preferred embodiment, the promotion of interaction of SCARECROW and SHORT ROOT in plants is to promote the combination of SHORT ROOT with the promoter of gene SCARECROW.

In a preferred embodiment, the elevation of expression or activity of SCARECROW and SHORT ROOT in plants, or the promotion of the interaction of SCARECROW and SHORT ROOT comprises: transforming genes SCARECROW and SHORT ROOT or expression constructs or vectors containing said gene into plants; improving expressive efficiency of genes SCARECROW and SHORT ROOT in plants by enhancers or tissue-specific promoters; increasing expressive efficiency of genes SCARECROW and SHORT ROOT in plants by enhancer; or exogenously adding the cis-element AT1 Box or Enhancer of gene SCARECROW in the promoter (pSCR) when the element is absent in plants.

In another aspect, the present disclosure provides a use of substances that improve the expression or activity of SCARECROW and SHORT ROOT in plants, or promote the interaction of SCARECROW and SHORTROOT, for improving traits of legumes or gramineous plants; wherein the improved traits are selected from the following group comprising: promoting the formation of infection threads, changing identity of cortical cells, improving abilities of cortical cells to respond to cytokinins, improving abilities of cortical cells to respond to rhizobial infection, promoting NIN-mediated plant spontaneous nodulation, promoting cortical cell division, promoting nodule organogenesis.

In another aspect, the present disclosure provides a method for screening substances for improving traits of legumes or gramineous plants, wherein the method comprises: (1) Adding candidate substance to the system containing protein SHORT ROOT and gene SCARECROW, wherein gene expression of the SCARECROW is driven by its promoter (pSCR); (2) Detecting the system to observe the interaction between SHORT ROOT and promoter of SCARECROW in the system of (1); if the candidate substance promotes the combination of two, or promotes the expression of pSCR in cortical cells, then the candidate substance is the substance improving traits of legumes or gramineous plants; wherein the improved traits are selected from the following group comprising: promoting the formation of infection threads, changing identity of cortical cells, improving abilities of cortical cells to respond to cytokinins and rhizobial infection, and promoting NIN-mediated plant spontaneous nodulation, cortical cell division and nodule organogenesis.

In a preferred embodiment, the cortical cells comprise: root cortex cells or epidermal cells.

In another preferred embodiment, the SCARECROW is from Medicago truncatula.

In another preferred embodiment, the protein SCARECROW has the amino acid sequences selected from the following group, comprising:
(a) a protein with the amino acid sequence as set forth in SEQ ID NO: 3.
(b) a protein formed by the one or more (such as 1-20; preferably 1-15; more preferably 1-10, such as 5, 3) amino acid residue substitution, deletion or addition in any amino acid sequence of SEQ ID NO:3 and derived from (a) with the function of protein (a); or
(c) a protein having more than 80% (preferably more than 85%; more preferably more than 90%; more preferably more than 95%, such as 98%, 99%) sequence identity to the protein with amino sequence in (a) and derived from (a) with the function of protein (a);
(d) a protein formed by active fragments defined in (a), or adding a tag, an enzyme cleavage sequence, and a reporter at both ends thereof.

In another preferred embodiment, the SHORT ROOT is from Medicago truncatula.

In another preferred embodiment, protein SHORT ROOT with amino acid sequences selected from the following group, comprising: (a′) a protein with the amino acid sequence as set forth in SEQ ID NO: 4. (b′) a protein formed by the one or more (such as 1-20; preferably 1-15; more preferably 1-10, such as 5, 3) amino acid residue substitution, deletion or addition in any amino acid sequence of SEQ ID NO:4 and derived from (a′) with the function of protein (a′); (c′) a protein having more than 80% (preferably more than 85%; more preferably more than 90%; more preferably more than 95%, such as 98%, 99%) sequence identity to the protein with amino sequence in (a′) and derived from (a′) with the function of protein (a′); or (d′) a protein formed by active fragments defined in (a′), or adding a tag, an enzyme cleavage sequence, and a reporter at both ends thereof.

Other aspects of the disclosure will be apparent to those skilled in the art based on the disclosure herein.

### BRIEF DESCRIPTION OF THE DRAWINGS

FIG. 1. AT1 box and Enhancer determine pMtSCR expression in cortical cells of Medicago truncatula and Arabidopsis thaliana. (A) ΔEn (deletion of Enhancer element), ΔAT1 (deletion of AT1 element) and ΔAT1ΔEn (deletion of both Enhancer and AT1 element) all significantly decreased pMtSCR expression in cortical cells of Medicago truncatula hairy root tips. (B) ΔEn, ΔAT1, ΔAT1/ΔEn all significantly decreased pMtSCR expression in root cortical cells of Arabidopsis thaliana. Red, PI staining; E: endodermis; C: cortex; QC: quiescent center. Scale bars, 20 μm.

FIG. 2. SCR has a conserved expression profile in legumes. (A) Analysis of the sequence of promoter reveals that legumes Medicago, Lotus, Glycine, Cicer, Pisum, Trifolium, and non-legumes Parasponia SCR gene promoters have both AT1-box and Enhancer, whereas the absence of at least one of these elements was observed in non-legumes Arabidopsis and Rice. (B) In situ hybridization shows cortical cell expression of SCR in Glycine, Lotus, Cicer, Pisum and Lupinus. Scale bars, 20 μm.

FIG. 3. SCR expressed in cortical cells are crucial for nodule symbiosis. (A) Numbers of nodules in Mtscr-1, Mtscr-2 mutant plants and wild-type (WT) at 7, 14, 21, and 28 days post-inoculation (dpi) with rhizobia. (B) Numbers of nodules at 21 dpi in wild-type and Mtscr-1 mutant hairy roots transformed with empty vector (EV), pMtSCR:MtSCR, pMtSCRΔEnΔAT1:MtSCR or pAtSCR:MtSCR. (C) Mtscr-1 plants stably transformed with pAtSCR:MtSCR could not restore the nodule-deficient phenotype. (D) Numbers of nodules at 21 dpi in Lotus hairy roots transformed with empty vector or pMtNRT1.3:LjSCR-SRDX (n≥12). (E) Representative images show that the number of nodules significantly reduced in LjSCR-SRDX transgenic hairy roots in Lotus. White arrows indicate nodules. (F) Numbers of nodules at 21 dpi in Glycine hairy roots transformed with empty vector or pMtNRT1.3:LjSCR-SRDX(n≥15). (G) Representative images show that the number of nodules significantly reduced in GmSCR-SRDX transgenic hairy roots in Glycine. White arrows indicate nodules. Asterisks in Figure (A), (D), (F) indicate significant differences by t-test (* P<0.05; ** P<0.01; ns, not significant); different letters (a/b) in Figure (B), (C) indicates significant differences (ANOVA, Duncan's multiple range tests; P<0.01). Center of the Boxes shows median quartile; Data points are represented by dots. n represents independent biological samples. These experiments were repeated twice with similar results. Scale bars, 1 mm (E, G).

FIG. 4. MtSCR and MtSCL23 function redundantly to some extent. (A) Maximum likelihood phylogenetic tree of SCR from A. thaliana; Oryza sativa (Q2RB59, A2ZAX5); Zea mays (NP_001168484); M. truncatula (Medtr7g074650); Selaginella moellendorffii (85562, 84762); Physcomitrella patens (Pp1s882_1V6.1, Pp1s85_139V6.1, Pp1s324_56V6.1) and its homologues from A. thaliana (AtSCL23, AtRGA1, AtSCL3, AtLAS/SCL18) and M. truncatula (Medtr4g076020, Medtr1g069725, Medtr3g065980, Medtr7g027190, Medtr8g442410). Branch support was obtained from 1,000 bootstrap repetitions. Evolutionary analyses were conducted in MEGA7 (B) Numbers of nodules in wild-type, Mtscr-1, and Mtscr-1/Mtscl23 at 7 days, 14 days, 21 days and 28 days post-inoculation (dpi) with Sm1021 (n≥19). Asterisks indicate significant differences by t-test (** P<0.01); Center of the Boxes shows median quartile; Data points are represented by dots. n represents independent biological samples.

FIG. 5. MtSCR interacts with MtSHR1/2. (A) Interactions between MtSCR and MtSHR1/2 in yeast two-hybrid assay. BD, binding domain; AD, activation domain. SD2, SD medium lacking leucine, tryptophan; SD4, SD medium lacking leucine, tryptophan, histidine and adenine. (B) Split luciferase complementation indicates an in vivo interaction between MtSCR and MtSHR1/2 in N. benthamiana leaves. Fluorescence signal intensity is indicated. JW771 and JW772, which were derived from pCAMBIA2300 (Gou, et al, 2011) by inserting Pro35S:nLUC or Pro35S:cLUC cassette, respectively, were used. (C) MtSCR co-immunoprecipitates with MtSHR1/2 in N. benthamiana. WB, western blot; IP, immunoprecipitation; CoIP, coimmunoprecipitation. MtSCR-HA and MtSHR1-FLAG or MtSHR2-FLAG were co-expressed in N. benthamiana leaves, respectively.

FIG. 6. MtSHR1/2 is located in the cells of stele, endodermis, cortex and epidermis, and MtSHR in cortical cells is involved in nodule symbiosis. (A) Representative images of GUS staining (roots stained in GUS staining solution for 60 min) in pMtSHR1/2:EGFP-GUS transgenic hairy roots show that MtSHR1 and MtSHR2 are expressed in the stele of Medicago truncatula root. (B) GUS staining for 60 min shows accumulation of MtSHR1/2 in the stele, epidermis, cortex, and endodermis. Note that GUS staining is limited to the stele in roots expressing MtSCR-GUS and AtSHR-GUS driven by the same promoter (bottom left of Figure B). (C) Immunostaining with Cy3 showing that MtSHR1 localized in the stele, epidermis, cortex and endodermis. (D) Numbers of nodules at 21 days after inoculation in Mtshr2 hairy roots transformed with empty vector, pLjUBQ:MtSHR1-SRDX, pMtSHR1:MtSHR1-SRDX, or pMtNRT1.3:MtSHR1-SRDX (n≥10). Scale bars, 100 μm (A, B); 50 μm (C).

FIG. 7. MtSHR-MtSCR determines cortical cell division. (A) Quantification of nodule primordia formed in wild-type hairy roots transformed with empty vector or pLjUBQ:MtSHR1-SRDX after spot inoculation with rhizobia for four days (n≥20). (B-C) Quantification (B) and representative images (C) of sections of nodule primordia formed in wild-type, Mtscr-1, Mtscr-1/Mtscl23 and Mtscr_1/pAtSCR:MtSCR stable transgenic roots three days after spot inoculation (n≥16). (D-E) Quantification of cortical cell division (D) and representative images (E) of wild-type, Mtscr-1, Mtscr-1/Mtscl23, and Mtscr-1/pAtSCR:MtSCR stable transgenic roots treated with 10 μM 6-BA for three days (n≥26). (F-G) Quantification of cortical cell division (F) and representative images (G) of empty vector and pLjUBQ:MtSHR1-SRDX transgenic hairy roots treated with 50 μM 6-BA for four days (n≥20). (H-J) Representative images (H), sections (I) and quantification (J) of spontaneous nodules by over-expression of NIN were significantly lower in Mtscr than in wild-type plants. Asterisks in Figure (J) indicate significant differences by t-test (** P<0.01); Center of the Boxes shows median quartile; Data points are represented by dots. Asterisks in Figure (A), (B), (D) and (F) indicate significant differences compared with controls by χ² test (* P<0.05; ** P<0.01; ns, not significant). Black arrows in Figure (C), (E) and (G) indicate cortical cell division; n represents independent biological samples. Scale bars, 50 μm (C, E, G, I); 1 mm (H).

FIG. 8. MtSHR1 overexpression in Medicago truncatula caused cortical cells divided into thick roots, and SHR overexpression in Arabidopsis thaliana and Rice promoted cortical cell divisions. (A-B) MtSHR1 overexpression in Medicago truncatula hairy roots induced cortical cell division and caused nodule-like structures without rhizobia inoculation (A), and sections of these roots(B). (C) Pairwise comparisons of all differentially expressed genes in roots overexpressing MtSHR1 and in wild-type roots 120 h after spot inoculation with rhizobia. Figure (C) shows that cortical cell divisions by overexpressing MtSHR1 are similar with that in roots 120 h after spot inoculation with rhizobia. RNA sequencing (RNAseq) data for 120 h after spot inoculation were obtained from Schiessl et al, 2019. (D) Cortex-specific over-expression of MtSHR1 caused cortical cell divisions. (E) In Arabidopsis thaliana, cortical cell divisions were induced within 24 h via 10 μM oestradiol treatment of stably transformed plant pG1090-XVE:AtSHR. Arrowheads indicate cortical cell divisions. En, endodermis; Co, cortex; Ep, epidermis. (F) Root sections of rice roots transformed with empty vector (EV) or MtSHR1-MtSCR. (G) Expression levels of MtSHR1. MtSCR compared with the reference gene Cyclophilin2. Asterisks indicate significant differences compared with control by t-test (** p<0.01). Error bars represent the standard deviation of three replicates. n represents independent biological samples. Data points are represented by dots. Scale bars, 1 mm (A), 100 μm (B, D, F) and 20 μm (E).

FIG. 9. MtSHR-MtSCR module is required for infection thread formation (A) Heat map showing changes in expression of genes in roots overexpressing MtSHR1 or 24 h and 120 h after spot inoculation with rhizobia for 24, 120 hours. Arrowheads, genes involved in infection thread formation. RNA sequencing (RNAseq) data for 24 h, 120 h after spot inoculation were obtained from Schiessl et al, 2019. (B) Numbers of infection threads and infection foci in wild-type (WT), Mtscr-1 and Mtscr-1Mtscl23 roots 7 days after inoculation with LacZ-marked rhizobia (n≥12). Different letters (a/b) indicate significant differences (ANOVA, Duncan's multiple range tests; P<0.05). (C) Numbers of infection threads and infection foci in empty vector and pLjUBQ:MtSHR1-SRDX transformed into Mtshr2 hairy roots 7 days after inoculation with LacZ-marked rhizobia (n≥14). Asterisks indicate significant differences compared with control by t-test (** p<0.01).

FIG. 10. The MtSHR-MtSCR module can be activated by symbiotic signals. (A) Quantative results show that expression of MtSCR but not MtSHR1/2 is induced in wild-type plants at 7 dpi during rhizobial symbiosis (n=5). (B) Relative expression of MtSCR in wild type, nsp1-1, nsp2-1 and nin-1 upon Nod factor treatment for 24 h. (C) Quantative results show that genetic impairment of MtSHR1/2 function significantly reduces the expression of MtSCR (at 7th day after inoculation with rhizobia). (D-E) GUS staining (D) and sections (E) show that MtSCR promoter and MtSHR1-GUS but not MtSHR1 promoter are expressed in nodule primordia. In Figure (A), (B) and (C), expression levels were normalized against the reference gene EF-1; Asterisks indicate significant differences compared with control by t-test (* P<0.05; ** P<0.01). Error bars represent the standard deviation of three replicates. n represents independent biological samples. Data points are represented by dots. Scale bars, 100 μm.

FIG. 11. Symbiotic signals accelerate MtSHR accumulation in cortical cells and then activate the expression of MtSCR. (A) Immunoblotting showing that MtSHR1-GUS (≈130 KD) accumulated in pMtSHR1:MtSHR1-GUS transgenic hairy roots after inoculation with rhizobia for 3 days, but the accumulation disappeared in nin-1 (n≥10). (B) GUS protein is not modified and accumulated upon rhizobia inoculation for 3 days in p35S:GUS transgenic hairy root. (C) GUS staining (10 min) of pMtSHR1:MtSHR1-GUS transformed hairy roots shows that rhizobia induced accumulation of MtSHR1-GUS in cortical and epidermal cells. These experiments were repeated twice with similar results. (D) ChIP-PCR shows that MtSHR1 associated with the promoter of an MtSCR fragment that does not overlap with the AT1-box (−1,604 bp to −1,615 bp) and enhancer (−1,632 bp to −1,638 bp). GFP-3×FLAG transformed hairy roots were used as control. (E) qPCR shows that overexpression of MtSHR1 triggers upregulation of MtSCR (n=5); Expression levels were normalized against the reference gene EF-1; These experiments were repeated in two independent experiments with similar results. Asterisks indicate significant differences compared with control by t-test (** p<0.01). Error bars represent the standard deviation of three replicates. n represents independent biological samples. Data points are represented by dots. Scale bars: 1 mm (root tips and root hairs) and 100 μm (root sections).

### DETAILED DESCRIPTION

Through the methods of genetics, cell biology and molecular biology, the inventors found that SHR-SCR is enriched in cortical cells of legumes. Overexpression of SHR-SCR in the cortex triggered cortical cell division, formed nodule-like structures without rhizobia infection, and induced the expression of genes related to nodule development and infection thread formation. The inventors also found that SHR protein can move to root cortical cells and epidermal cells to control early cortical cell division in nodule development, and SHR-SCR of cortical cells determines the division potential of cortical cells. The new discovery of the present disclosure provides a new way for the improvement of plant nodule traits.

### Genes and Plants

As used herein, the “SCARECROW (SCR) gene” or “SCR polypeptide” refers to an SCR gene or polypeptide from Medicago truncatula or the SCR gene or polypeptide that is homologous to a Medicago truncatula-derived gene or polypeptide and with substantially the same structural domains and substantially the same functions.

As used herein, the “SHORT ROOT (SHR) gene” or “SHR polypeptide” refers to an SHR gene or polypeptide from Medicago truncatula or the SCR gene or polypeptide that is homologous to a Medicago truncatula-derived gene or polypeptide, with substantially the same structural domains and substantially the same functions.

In the present disclosure, the SCR polypeptide and SHR polypeptide also comprise their fragments, derivatives and analogs. As used herein, the term “fragment” “derivative” or “analog” refers to a protein fragment that essentially maintains the functions or activities of the polypeptides, and may be a protein (i) substituted by one or more conservative or non-conservative amino acid residues (preferably conservative amino acid residues), and such substituted amino acid residues may or may not be encoded by the genetic code, or (ii) with a substitution group in one or more amino acid residues, or (iii) formed by an additional amino acid sequence fused to the protein sequence, and so on. According to the teaching herein, these fragments, derivatives, and analogues belong to the common knowledge to those skilled in the art. Biologically active fragments of the SCR polypeptides and SHR polypeptides can all be applied to the present disclosure.

In the present disclosure, the term “SCR polypeptide” refers to a protein with the sequence of SEQ ID NO: 3 and biological activities of SCR polypeptide. The term also comprises variants of the sequence of SEQ ID NO: 3 with same functions as the SCR polypeptide. The variants may include (but are not limited to): deletion, insertion and/or substitution of one or several (usually 1-50, preferably 1-30, more preferably 1-20, most preferably 1-10, even more preferably 1-8, 1-5) amino acids, and addition or deletion of one or several (usually within 20, preferably within 10, more preferably within 5) amino acids at the C-terminal and/or N-terminal. For example, in the art, substitution with amino acids of approaching or similar properties generally does not alter the functions of a protein. For another example, the addition of one or several amino acids to the C-terminal and/or N-terminal also generally does not alter the function of a protein.

In the present disclosure, the term “SHR polypeptide” refers to a protein with the sequence of SEQ ID NO: 4 and biological activities of SHR polypeptide. The term also comprises variants of the sequence of SEQ ID NO: 4 with same functions as the SHR polypeptide. The variants may include (but are not limited to): deletion, insertion and/or substitution of one or several (usually 1-50, preferably 1-30, more preferably 1-20, most preferably 1-10, even more preferably 1-8, 1-5) amino acids, and addition or deletion of one or several (usually within 20, preferably within 10, more preferably within 5) amino acids at the C-terminal and/or N-terminal.

The present disclosure also comprises a polynucleotide (gene) encoding the polypeptide, such as the polynucleotide of the nucleotide sequence shown in SEQ ID NO: 1 or a degenerate sequence thereof, which can encode the SCR polypeptide of SEQ ID NO: 3; the polynucleotide of the nucleotide sequence shown in SEQ ID NO:2 or a degenerate sequence thereof, which can encode the SHR polypeptide of SEQ ID NO:4.

It should be understood that although the SCR gene and SHR gene of the present disclosure are preferably obtained from legumes, especially Medicago truncatula, but other genes or its degenerate forms obtained from other plants that are highly homologous to the SCR gene and SHR gene of Medicago truncatula (with more than such as 80%, 85%, 90%, 95%, or even 98% sequence identity) are also within the scope of the present disclosure. Methods and tools for aligning sequence identity are also well known in the art, such as BLAST.

Vectors comprising said coding sequences, as well as host cells genetically engineered with said coding sequences of said vectors or polypeptides, are also included in the present disclosure. Methods known to those skilled in the art can be used to construct suitable expression vectors.

Host cells are usually plant cells. For transforming plants, methods such as Agrobacterium transformation or biolistic transformation can generally be used, such as leaf disk method, rice immature embryo transformation method, and so on; preferably Agrobacterium transformation. Transformed plant cells, tissues or organs can be regenerated into plants using conventional methods to obtain plants with altered traits relative to the wild type.

As used herein, the plants comprise but are not limited to plants selected from the group consisting of: plants expressing the gene SCARECROW; rhizobial plants; gramineous plants and/or cruciferous plants.

As used herein, the term “rhizobial plants” mainly refers to plants that can be invaded by rhizobia and stimulated to form nodules in roots. The “rhizobial plants” can include both leguminous rhizobial plants and non-leguminous rhizobial plants. Preferably, the “rhizobial plants” is “legumes”.

As used herein, the “nodule-like plant” refers to a plant with nodule-like structures.

The rhizobial plants preferably comprise legumes; more preferably, comprise (but not limited to): edibles such as soybeans, broad beans, peas, mung beans, adzuki beans, cowpeas, kidney beans, hyacinth beans, pigeonpeas, peanuts, etc.; feeds such as alfalfa, astragalus smicus, broad beans, clover, etc.; timbers such as Albizia julibrissin, Dalbergia hupeana, erythrophloeum ferdii, adzuki beans, Sophora japonica, etc.; dyes such as Indigofera pseudotinctoria, flos Sophorae, Indigofera tinctoria, sappanwood, etc.; resins such as acacia gum, astragalus gum, dopal gum, etc.; fibers such as Crotalaria juncea and Pueraria lobata, etc.; oilseeds such as soybeans, peanuts, etc. It should be understood that under the guidance of the technical solutions of the present disclosure, those skilled in the art can easily think of changing the types of various leguminous crops to achieve the same or similar technical effects, and these changes are also included in the present disclosure.

The gramineous plants; preferably comprise: rice, barley, wheat, oats, rye, corn, sorghum.

### Cis-Elements and Applications Thereof

During the detailed analyses of the SCR gene, the inventors unexpectedly found that when the AT1 Box (AT1 for short) and Enhancer (En for short) are deleted, the expression activity of the SCR promoter in cortical cells is significantly reduced or lost. It indicated that cis-elements AT1 and Enhancer govern the expression of MtSCR promoter in root cortical cells.

The cis-elements were different from each other on positions upstream of the SCR promoter in different species of legumes, but their functions remain the same, which shows that they are highly conserved in legumes.

Based on this new discovery of the present inventors, the cis-elements can be used as molecular markers to directionally-screen plants, or to identify the cortical cell division ability or cortical biomass of plants.

Therefore, the present disclosure provides a method for directionally screening plants with normal cortical cell division ability or cortical biomass, comprising: analyzing the promoter of plants' gene SCARECROW (pSCR); wherein, if cis-elements AT1 Box and Enhancer are present at the same time, it indicates that the gene SCARECROW is expressed normally and formation of infection threads, abilities of cortical cells to respond to cytokinins and rhizobial infection, NIN-mediated plant spontaneous nodulation, cortical cell division or nodule organogenesis are normal.

The present disclosure also provides a method for identifying cortical cell division ability or cortical biomass (including cortical thickness) of plants, comprising: analyzing the promoter of plants' gene SCARECROW (pSCR); wherein, if cis-elements AT1 Box and Enhancer are present at the same time, it indicates that formation of infection threads, abilities of cortical cells to respond to cytokinins and rhizobial infection, NIN-mediated plant spontaneous nodulation, cortical cell division or nodule organogenesis are normal; if either AT1 Box or Enhancer is absent, it indicates that formation of infection threads, abilities of cortical cells to respond to cytokinins and rhizobial infection, NIN-mediated plant spontaneous nodulation, cortical cell division or nodule organogenesis are abnormal.

The present disclosure provides a method for screening substances for improving traits of legumes or gramineous plants, comprising: (1) Adding candidate substance to the system containing SHR and gene SCR, wherein gene expression of the SCR is driven by its promoter (pSCR); (2) Detecting the system to observe the interaction between SHR and promoter of SCR in the system of (1); if the candidate substance promotes the combination of two, then the candidate substance is the substance improving traits of legumes or gramineous plants; wherein the improved traits are selected from the following group comprising: promoting the formation of infection threads, improving abilities of cortical cells to respond to cytokinins and promoting NIN-mediated plant spontaneous nodulation, cortical cell division and nodule organogenesis.

The methods for screening substances acting on a protein or gene or its specific region as a target are well known to those skilled in the art, and these methods can be used in the present disclosure. The candidate substances can be selected from: peptides, polymeric peptides, peptidomimetics, non-peptide compounds, carbohydrates, lipids, antibodies or antibody fragments, ligands, small organic molecules, small inorganic molecules, nucleic acid sequences, and the like. Based on the type of substances to be screened, it is clear to those skilled in the art how to select a suitable screening method.

After large-scale screening, a class of potential substances with specific functions on the complex of interacted SHR and SCR gene promoters and regulatory effects can be obtained.

In above directionally screening and identification, some technical means known in the art can be used. The method for obtaining DNA of the sample to be tested is well known to those skilled in the art, such as the traditional phenol/chloroform/isoamyl alcohol method, or some commercially available DNA extraction kits, which are well known to those skilled in the art. The polymerase chain reaction (PCR) technology is well known to those skilled in the art, and its basic principle is a method of enzymatically synthesizing specific DNA fragments in vitro. The methods of the present disclosure can be carried out using conventional PCR techniques.

### Applications of Plant Improvement

Based on new findings of the inventors, the present disclosure provides a use of substances that improve the expressions or activities of SCR and SHR in plants, or promote the interaction of SCR and SHR, for improving traits of plants; wherein the improved traits are selected from the following group comprising: promoting the formation of infection threads, improving abilities of cortical cells to respond to cytokinins and rhizobial infection, and promoting NIN-mediated plant spontaneous nodulation, cortical cell division and nodule organogenesis.

Symbiosis of legumes and rhizobia begins with rhizobia infection of hairy roots, and a special tubular channel called an infection thread is formed in the infected hairy roots, with rhizobia expanding in the infection thread, further other cells are infected. The studies of the present inventors found that some genes affecting the infection threads were activated in the overexpressed plant material, thus revealing that SHR-SCR was involved in the formation of the infection threads, which was confirmed by further experiments and observations. In the absence of rhizobia infection, the cortical cells of legumes can specifically respond to cytokinins and divide into pseudonodule. The inventors found that the SHR-SCR determines the root cortical cell division respond to cytokinins. These findings of the present disclosure have not been previously studied in the art.

It should be understood that according to the experimental data and regulatory mechanisms provided by the present disclosure, various methods well known to those skilled in the art can be used to regulate the expression of the SCR and SHR, and these methods are all included in the present disclosure.

In the present disclosure, substances that increase the expression or activity of SCR and SHR in plants, or promote the interaction between SCR and SHR include promoters, agonists, and activators. The “up-regulation”, “improvement” or “promotion” includes “up-regulation”, “promotion” of protein activities or “up-regulation”, “improvement” and “promotion” of protein expressions. Any substance that can increase the activity of SCR and/or SHR protein, increase the stability of SCR and/or SHR gene or the encoded protein thereof, upregulate the expression of SCR and/or SHR gene and increase the effective time of SCR and/or SHR protein can be used in the present disclosure as useful substances for up-regulating SCR and/or SHR genes or the encoded proteins thereof. They can be chemical compounds, small chemical molecules, biomolecules. The biomolecules can be nucleic acids (including DNA, RNA) or proteins.

As another embodiment of the present disclosure, there is also provided a method for up-regulating the expression of SCR and/or SHR genes or encoded proteins thereof in plants, wherein the method comprising: transferring SCR and/or SHR genes, constructs or vectors of the encoding protein thereof into the plants.

The main advantages of the present disclosure are:

The inventors have deeply studied the mechanisms of SHR-SCR in cortical cell division of rhizobial symbiosis in legumes and found that SHR-SCR controls the cortical cell division potential of rhizobial symbiosis and also has important applicable value for modifying non-legume root cortical cell identity and finally realizing nodule organogenesis of non-legumes.

The present disclosure provided a novel approach for identifying the traits of plants, thereby providing a feasible method for plant identification and an effective tool for plant breeding and screening.

The present disclosure can identify interesting characters of plants at the early stage of planting, which brings great convenience to plant breeding.

The disclosure is further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3^{rd} ed. Science Press, 2002) or followed the manufacturer's recommendation.

Unless otherwise defined, all professional and scientific terms used herein have the same meanings as familiar to those skilled in the art. In addition, any methods and materials similar or equivalent to those described can be used in the present disclosure. Methods and materials for preferred embodiments described herein are provided for illustrative purposes only.

### I. Materials and Methods

### 1. Experimental Materials

### 1.1. Plant Materials

According to different experimental requirements, the inventors selected Medicago truncatula ecotypes Jemalong A17 and R108 respectively as wild types for all hairy root transformation experiments. In the present disclosure, the Medicago truncatula R108 Tnt1 insertion lines of Mtscr-1 (NF11026), Mtscr-2 (NF20550), Mtshr2(NF13823) were obtained from the Noble Foundation Tnt1 database (http://medicago-mutant.noble.org/mutant/database.php) and all are R108 backgrounds.

MtSCR and MtSCL23 function redundantly to control the formation of roots and nodules. Therefore, the inventors obtained Mtscr-1/Mtscl23 double mutants. Plant materials of Mtscr-1/Mtscl23 double mutants: Mtscr-1/Mtscl23 double mutants was obtained by crossing Mtscl23 (NF9220) mutant as male parent and Mtscr-1 mutant as female parent.

The plants were placed in a growth room at 24° C. with 16 h light/22° C., 8 h dark.

### 1.2. Strains and Vectors

E. coli for cloning: DH5α, CCDB3.1;
Agrobacterium: Arqul (AR);
Rhizobium: Sm1021;
Entry Vector: pENTR/SD/D-Topo (Invitrogen);
Vector pG1090: obtained from Prof. Wu Shuang, Fujian Agriculture and Forestry University.
Plant expression vector: pK7WG2R was obtained from the laboratory of Dr. Giles Oldroyd, University of Cambridge, UK.
pK7WG2R-pMtNRT1.3 (root cortical cell-specific promoter): obtained from the laboratory of Dr. Giles Oldroyd, University of Cambridge, UK.
pK7WG2R-pLjUBQ (plant ubiquitous promoter): obtained by inserting the promoter pLjUBQ (37766-38887 bp in GenBank accession number AP009383.1) into the 6242/7309 bp site of pK7WG2R.
pK7WG2R-pMtSHR1 (stele-specific promoter): obtained by inserting the promoter pMtSHR1 (Medtr5g015490, 2939 bp fragment before ATG of the promoter) into the 6242/7309 bp site of pK7WG2R.
pK7WG2R-pMtSHR2 (stele-specific promoter): obtained by inserting the promoter pMtSHR2 (Medtr4g097080, 3161 bp fragment before ATG of the promoter) into the 6242/7309 bp site of pK7WG2R.
pK7WG2R-pAtSCR (endothelial cell-specific promoter): obtained by inserting the promoter pAtSCR (At3g54220, 1686 bp fragment before ATG of the promoter) into the 6242/7309 bp site of pK7WG2R.
pG1090-XVE:AtSHR was obtained from Prof. Wu Shuang, Fujian Agriculture and Forestry University.
Rice expression vector: pZmUBI:SHR-TNOS was inserted into the pYL322-d1 vector, p35S:SCR-PolyA was inserted into the pYL322-d2 vector, and then recombining pYL322-d1-pZmUBI:SHR-TNOS with destination vector pYLTAC380H to obtain pYLTAC380H-pZmUBI:SHR-TNOS; then recombining pYL322-d2-p35S:SCR-PolyA with pYLTAC380H-pZmUBI:SHR-TNOS to obtain pYLTAC380H-pZmUBI:SHR-TNOS-p35S:SCR-PolyA (that is the construction of over-expressing SHR and SCR).
The expression vector for identifying AT1 Box and Enhancer was pBGWFS7: pMtSCR (2899 bp) or deletion of AT1 (ΔAT1), En (ΔEn) or both (ΔAT1ΔEn) were first ligated to the intermediate vector pENTR/SD/D-Topo, and then recombining into pBGWFS7 with the EGFP-GUS reporter. And the EGFP-GUS reporter was inserted after the promoter. Wherein, the pMtSCR (2899 bp) mutant promoters comprise: deletion of AT1 Box (ΔAT1), deletion of Enhancer (ΔEn), or simultaneous deletion of AT1 and En (ΔAT1ΔEn).

### 2. Experimental Methods

### 2.1. Plasmid Construction

Firstly, primers MtSHR-F/MtSHR-R and KOD enzyme (high-fidelity DNA polymerase, purchased from Toyobo) were used to amplify with Medicago truncatula gDNA. After PCR products recovered, they were digested with BamHI and EcoRI and then fused to the pENTR vector for transformation. The positive clones were identified by Escherichia coli and the plasmid DNA was extracted. After sequencing, a recombinant plasmid carrying the MtSHR1 gene was obtained and verified.

The primer sequences are as follows:

MtSHR-F:
(SEQ ID NO: 25)
CGGGATCCTATGGATACATTGTTTAGACTTG;
MtSHR-R:
(SEQ ID NO: 26)
CCGGAATTCCTCAAGGCCTCCATGCACTGGC.

Construction of MtSHR1-SRDX suppressor: dominant suppressor SRDX was used and the SRDX sequence was linked to the 3′ end of the MtSHR1 gene.

SRDX sequence: 5′>ctagatctggatctagaactccgtttgggtttcgcttaa>3′ (SEQ ID NO: 27).

MtSHR1-SRDX was recombined downstream of the pK7WG2R-pMtNRT1.3, pK7WG2R-pMtSHR1 or pK7WG2R-pLjUBQ promoter using LR enzyme (purchased from Invitrogen). The obtained recombinant plasmids were transformed into E. coli to identify positive clones, and DNA of the plasmids were extracted for use.

Using the pK7WG2R-pAtSCR plasmid carrying the AtSCR promoter (At3g54220, a 1686 bp promoter fragment upstream of the ATG), MtSCR CDS was inserted downstream of the promoter to obtain a recombinant plasmid expressing MtSCR.

2.2. Agrobacterium rhizogenes Transformation
Preparation of Agrobacterium rhizogenes Competent Cells

### 1) Media and Solutions

Ultrapure water, LB medium, 10% glycerol (v/v).

### 2) Preparation of Competent Cells

Step 1: Preserved strains were used, then streaked on LB plates containing corresponding antibiotics and cultured at 28° C. for 24-48 hrs.
Step 2: A single clone was picked into 3 mL LB liquid medium, shaked overnight at 28° C., then inoculated into antibiotic free-LB medium at 1:100 and cultivated at 28° C. an 200 rpm until OD600=0.5-1.0.
Step 3: Ice bath for 10 min, then centrifuged at 2,500 g for 10 min at 4° C.
Step 4: After the supernatant removed, cells were suspended lightly with 5 mL ice-cold ultrapure water, then added 200 mL ice-cold ultrapure water, centrifuged at 2,500 g for 10 min at 4° C.
Step 5: Repeated Step 4 once.
Step 6: After the supernatant removed, cells were suspended lightly with 5 mL ice-cold 10% glycerol, then added 200 mL ice-cold 10% glycerol, centrifuged at 2,500 g for 10 min at 4° C.
Step 7: Repeated Step 6 once.
Step 8: The supernatant was completely removed, then added 50 mL 10% glycerol. The cells were resuspended and dispensed with 200 μL/tube.
Step 9: Quick-freeze in liquid nitrogen and store in a −80° C. refrigerator for later use.
   The Transformation of Expression Vector into Agrobacterium rhizogenes (AR)
Step 1: Clean electric shock cup and dry it for later use. At the same time, stored competent cells were used and melted on ice.
Step 2: 0.5-1 μL of the plasmids were pipetted into the competent cells, and mixed by gently pipetting.
Step 3: The competent solution containing the plasmids were transferred into the electric shock cup and shocked at 1.6-1.8 kV.
Step 4: Immediately after the electric shock was completed, the transformants were washed out into an EP tube with 600 μL antibiotic free-liquid LB, and recovered at 28° C. and 220 rpm for 1 h.
Step 5: Centrifuged at 4,000 rpm for 2 min, then the excess supernatant was aspirated with 50 μL left to resuspend the cells and spread on LB plates containing corresponding antibiotics.
Step 6: Inverted culture at 28° C. for 24-48 h and single clones were picked for identification.
   Preparation of Medicago truncatula Transgenic Plants
   a) Germination of Medicago truncatula Seeds
Step 1: Same size, undamaged Medicago truncatula seeds were selected and placed in a 2 mL EP tube (about 100 seeds per tube).
Step 2: 1 mL concentrated sulfuric acid was added and mixed sufficiently until small black spots appeared on most seed coats, then immediately removed the concentrated sulfuric acid and rinsed with water 5 times.
Step 3: Excess water was sucked off, then 1 ml 10% NaClO was added to each tube, inverted and mixed for 2-3 min.
Step 4: Aspirate off the NaClO and rinse the seeds with sterile water, repeat 5 times.
Step 5: Spreaded the seeds on a 1% Agar plate and inverted the plate at 4° C. for 2 days in the dark.
Step 6: One day before root cutting transformation, the seeds were invertly cultured at 24° C. in the dark for about 16 hrs to germinate.

### b) Preparation of Bacterial Solution and Infective Transformation

Step 1: Stored Arqual was streaked on plates in advance to activate the strain.
Step 2: A single clone was picked in 3 mL TY medium (with antibiotics) and cultured overnight at 30° C., 220 rpm until OD₆₀₀>1.5.
Step 3: The night before root cutting, the strain was inoculated into a new 30 mL TY medium (with antibiotics) at 1:1000, and cultivated overnight at 30° C., 220 rpm to OD₆₀₀=0.6-1.0.
Step 4: The cells were collected by centrifugation at 4,000 rpm for 10 min, then resuspended with 5 mL of antibiotic free-TY medium and transferred to a small dish for use.
Step 5: The germinated Medicago truncatula seeds were removed with an appropriate amount of sterile water added to keep the seeds moist.
Step 6: By using sterile tweezers, the seeds were placed on the plate cover. Root tips (about 3-5 mm from the cotyledon knot) were cut off and placed in a small dish containing bacterial liquid.
Step 7: After cutting the Medicago truncatula seedlings to be transformed, the seeds containing bacterial liquid from the wound were transferred into FP medium.
Step 8: After cultured in an incubator for 7-10 days, all grown roots that are close to the stem were cut off, wherein swollen part at the bottom of the radicle cannot be cut off.
Step 9: The cut Medicago truncatula seedlings were transferred into MFP medium, and cultivated in an incubator for 3-4 weeks.

### c) Screening Identification and Phenotypic Statistics

Step 1: After above transformed seedlings were cultured for 3-4 weeks, the seedlings were removed from medium and the medium was cleaned up.
Step 2: Non-positive roots were excised by fluorescent markers of vectors using a stereoscope, and then positive roots were used for oscillating slice.

### Oscillating Slice

### 1) 3% Low Melting Point Agarose

low melting point agarose: 0.6 g
ddH₂O: 20 mL

NOTE: The concentration of low melting point agarose should be between 2%-3%. When heating and dissolving in a microwave oven, heat it for 30 sec for the first time, and then heat it at intervals after it boils. Each heating should not exceed 7 sec, otherwise it will be easy to spray.

### 2) Embedding and Sectioning

Step 1: Fresh and relatively young roots were selected and cut into small pieces of about 3-4 mm. Avoid pulling, squeezing and damaging the tissue during the collection process.
Step 2: Dissolved low agarose solution was added into a small container (such as the lid of the electric shock cup), then toilet paper was used to absorb excess water adhering on the material, and placed flatly on the bottom of the container with low melting point agarose (For lid of the electric shock cup, 5 roots can be arranged in parallel with a little space between the samples. After the embedding was completed, 5 materials can be sliced together) Wait for solidify at room temperature.
Step 3: Solidified material was directly used for slicing or wrapped in plastic wrap and temporarily stored at 4° C. The Leica VT1200S oscillating microtome was used for slicing, with forward speed 1 mm/sec, amplitude 1 mm, and slice thickness 50 μm. If the material is relatively hard, the amplitude can be appropriately increased and the forward speed can be decreased. On the contrary, if the material is relatively young, the amplitude can be appropriately decreased, with forward speed increased.
Step 4: The slices can be directly placed on a glass slide for observation or placed in a 2 mL EP tube for temporary storage at 4° C.

### 2.3. Data Analysis Platform and Software

BLAST analysis of sequence: NCBI online analysis platform.
Alignment analysis of Sequence: SerialCloner 2.6.1 software.
Primer design: Primer Premier 5.0 software.
Microscopic observation: Zeiss Axio Scope A1.

### II. Example

### Example 1. SCR is Conservatively Expressed in Cortical Cells of Legumes

### 1. MtSCR is Widely Expressed in Root Cortical Cells and Epidermal Cells.

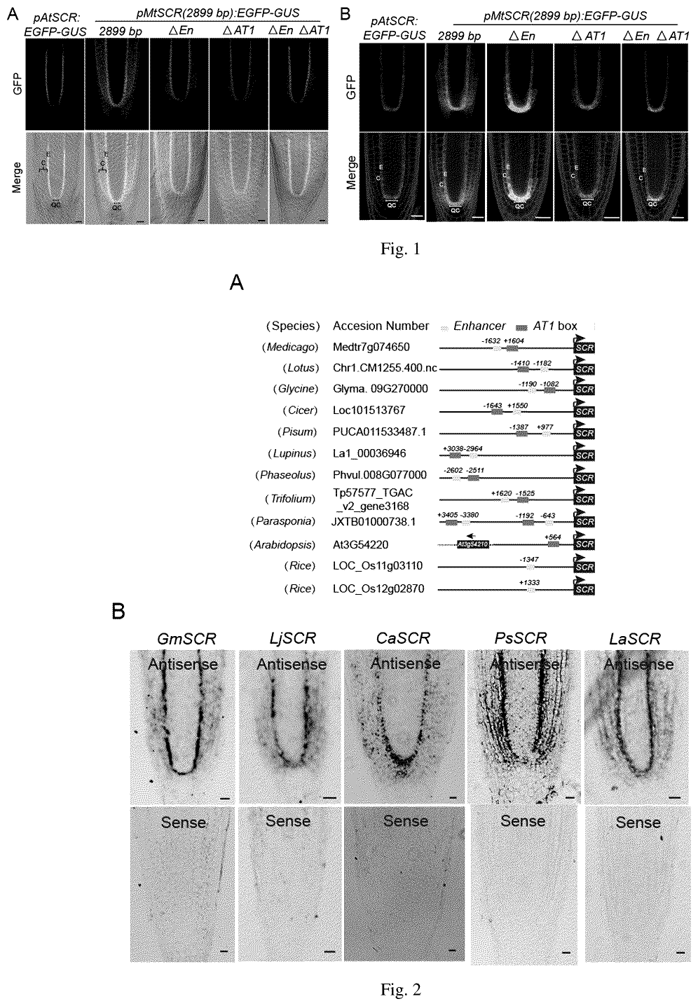

The inventor found that Medicago truncatula SCARECROW (MtSCR) gene is expressed not only in the cells of quiescent center and endodermis, but also in cortical cells and epidermal cells of Medicago truncatula roots. Different to MtSCR, Arabidopsis thaliana AtSCR is specifically expressed in the quiescent center and endodermis of Medicago truncatula roots (FIG. 1A-1B).

### 2. AT1 and Enhancer Control the Expression of MtSCR Promoter in Root Cortical Cells.

The inventor examined a series of MtSCR promoter (2899 bp upstream of MtSCR ATG; called pMtSCR (2899 bp)) truncation experiments, combined with a cis-element predictive analysis (http://plantpan2.itps.ncku.edu.tw/) and found that deletion of both AT1 Box (AT1 for short) and Enhancer (En for short) largely decreased gene expression in Medicago truncatula cortical cells (FIG. 1A), while abolished expression completely in Arabidopsis thaliana cortical cells (FIG. 1B). It indicated that cis-elements AT1 and Enhancer control the expression of MtSCR promoter in root cortical cells

### 3. AT1 and Enhancer May Function Conservatively in Legumes.

The inventors further studied and found that other legumes and the only non-legume Parasponia SCR gene promoters harbour closely located AT1 and Enhancer elements, whereas the absence of at least one of these elements, were observed in non-legumes such as Arabidopsis thaliana and Medicago truncatula (FIG. 2A). For a variety of legumes, stable SCR expression was detected in the endodermis and cortex of Medicago truncatula, Lotus, Glycine, Cicer, Pisum, Lupinus, and so on.

In order to explore whether SCR of other legumes exists in cortical cells, the inventors did in situ hybridization experiments and found that in legumes Lotus (LjSCR), Glycine (GmSCR), Cicer (CaSCR), Pisum (PsSCR) and Lupinus (LaSCR), SCR were expressed in root cortical cells (FIG. 2B).

Table 1 shows the positions of Enhancer and AT1 in the SCR promoters of legumes and non-legumes Parasponia.

TABLE 1*

| | Enhancer | AT1 |
|---|---|---|
| Medicago | −1632 (GAATTAC | +1604 (AAAAAAAATATT |
| truncatula | (SEQ ID NO: 5)) | (SEQ ID NO: 15)) |
| Lotus | −1182 (GAATTTC | −1410 (AATATTTTTTAT |
| | (SEQ ID NO: 6)) | (SEQ ID NO: 16)) |
| Glycine | −1190 (GACTTCC | −1082 (AATTTTTTTTTA |
| | (SEQ ID NO: 7)) | (SEQ ID NO: 17)) |
| Cicer | +1550 (GAAAATC | −1643 (AATATTTTTTTT |
| | (SEQ ID NO: 8)) | (SEQ ID NO: 18)) |
| Pisum | +977 (GAAAATC | −1387 (AATATATTTTTT |
| | (SEQ ID NO: 9)) | (SEQ ID NO: 19)) |
| Lupinus | −2964 (GATTTGC | +3038 (GATACAAATATT |
| | (SEQ ID NO: 10)) | (SEQ ID NO: 20)) |
| Phaseolus | −2602 (GAATTAC | −2511 (AATATTTATTT |
| | (SEQ ID NO: 11)) | (SEQ ID NO: 21)) |
| Trifolium | +1620 (GAAAGTC | −1525 (AATATTTTATTT |
| | (SEQ ID NO: 12)) | (SEQ ID NO: 22)) |
| Parasponia | −3380 (GATTTTC | +3405 (AACGAAAATATT |
| | (SEQ ID NO: 13)) | (SEQ ID NO: 23)) |
| | −643 (GATTTTC | −1192 (AATATTTTTTTT |
| | (SEQ ID NO: 14)) | (SEQ ID NO: 24)) |

*In the table, “+” indicates that the cis-element is located in the sense strand (5′→3′); “−” indicates that the cis-element is located in the antisense strand (3′→5′).
Above results suggest that the cis-elements AT1 and Enhancer in the SCR promoter are conserved in legumes to govern SCR expression in root cortical cells.

### Example 2. MtSCR Participates in Rhizobial Symbiosis

1. MtSCR participates in rhizobial symbiosis Medicago truncatula Tnt1 insertion lines of Mtscr-1 (NF11026) and Mtscr-2 (NF20550) were obtained by the inventors and grew in the environment of 24° C., 16 h light/22° C., 8 h darkness; Wide type Medicago truncatula (WT) was used as a control.

Plants were grown for 3 days before Sm1021 inoculation. After that, nodule growth of the plants was measured at 7th, 14th, 21st and 28th day post-inoculation (dpi), respectively.

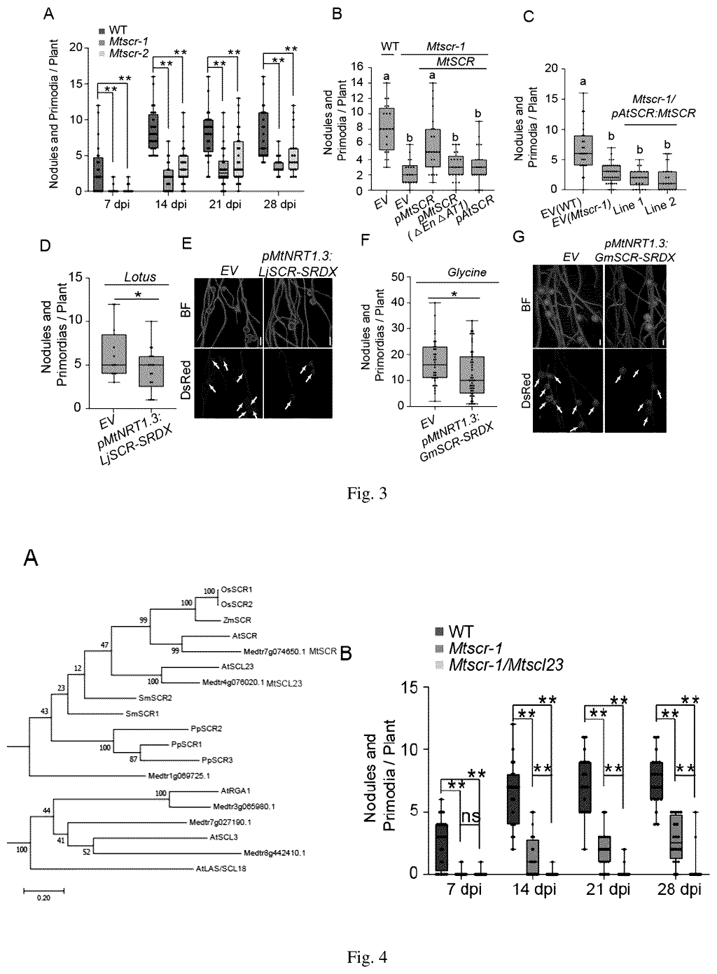

The results are shown in FIG. 3A, the wild-type Medicago truncatula can produce nodules normally, while the Tnt1 insertion mutants Mtscr-1 (NF11026) and Mtscr-2 (NF20550) of Medicago truncatula produced few nodules at 7 dpi, also with much lower amounts of nodules produced at 14 dpi, 21 dpi and 28 dpi compared with the wild type.

Above results indicate that the MtSCR gene in Medicago truncatula is involved in rhizobial symbiosis, and the decrease of its expression or activity will cause defects in root nodule generation.

2. SCR in Medicago truncatula Cortical Cells is Involved in Rhizobial Symbiosis of Medicago Truncatula.

To investigate whether SCR in Medicago truncatula cortical cells is involved in rhizobial symbiosis, the inventors firstly used the differential expression of AtSCR promoter, MtSCR promoter and MtSCR (ΔEnΔAT1) promoter in Medicago truncatula cortical cells (as shown in FIG. 1A) to perform Mtscr-1 hairy root transformation and complementation. The inventors found that only MtSCR promoter rescued the nodulation phenotype of Mtscr-1 (FIG. 3B) and deletion both cis-elements at the same time could not rescued the nodulation phenotype of Mtscr-1. It indicates that expression of MtSCR in cortical cells is a complementary key for the nodulation phenotype.

For verification, the inventors obtained pAtSCR:MtSCR (Arabidopsis thaliana AtSCR promoter-driven MtSCR expression) stably transformed complementary plants through tissue culture. Phenotypic analysis found that nodulation phenotype of the mutants could not be rescued (FIG. 3C), further indicating that SCR in Medicago truncatula cortical cells is essential for Medicago truncatula rhizobial symbiosis.

### 3. SCR of Cortical Cells in Legumes Participates in Rhizobial Symbiosis.

The inventors used the root cortical cell-specific promoter (pMtNRT1.3) in Glycine max (Gm) and Lotus japonicas (Lj), respectively, to cortical cell-specific dominantly inhibit SCR function (pMtNRT1.3:SCR-SRDX) and found that when the SCR of cortical cells was function-defective, the number of nodules in Glycine max and Lotus japonicas was significantly reduced (FIG. 3D-G), suggesting that the participation of SCR of cortical cells in rhizobial symbiosis is conserved in legumes.

### 4. MtSCL23 and MtSCR Function Redundantly in Controlling Root Nodulation.

Evolutionary analyses of the present disclosure found that MtSCL23 and MtSCR function redundantly to some extent (FIG. 4A). Mtscr-1/Mtscl23 double mutants was obtained by crossing Mtscl23 (NF9220) and Mtscr-1 by the inventors. Phenotypic analyses revealed that Mtscr-1/Mtscl23 was severely defective in nodulation, and most Mtscr-1/Mtscl23 could not form nodule (FIG. 4B), indicating that MtSCL23 and MtSCR function redundantly to some extent in controlling root nodulation.

### Example 3. MtSHR1/2 is Present in Root Cortical Cells of Medicago truncatula and is Required for Nodule Symbiosis

### 1. The Interaction of MtSHR-MtSCR

Findings in Arabidopsis thaliana suggest that MtSHR-MtSCR usually functions as a complex. Therefore, the inventors firstly found that the Medicago truncatula genome encodes two SHR homologues-Medtr5g015490 and Medtr4g097080, here named MtSHR1 and MtSHR2, respectively.

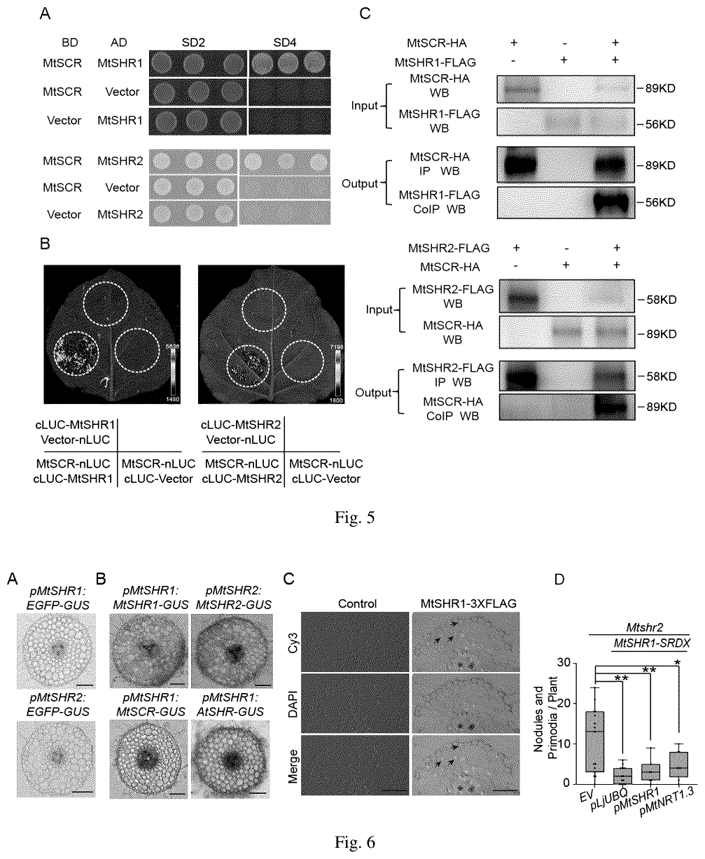

The inventors then verified the interaction of MtSHR1/2-MtSCR by conventional yeast two-hybrid assay, split luciferase complementation and coimmunoprecipitation (FIG. 5A-C).

### 2. MtSHR1/2 is Present in Cortical Cells and Epidermal Cells of Medicago Truncatula.

The inventors found that the MtSHR1 and MtSHR2 promoters were specifically expressed in the stele in the hairy roots of Medicago truncatula (FIG. 6A), but the MtSHR1 and MtSHR2 proteins could be present in cortical cells and epidermal cells of the hairy roots of Medicago truncatula (FIG. 6B-C), while corresponding Arabidopsis thaliana AtSHR protein and MtSCR protein are only present in the stele although using the same MtSHR1 promoter to initiate expression (FIG. 6B).

Above results indicated that, compared with the Arabidopsis thaliana AtSHR protein, the Medicago truncatula MtSHR1 and MtSHR2 proteins have stronger mobility and could be present in the root cortical cells and epidermal cells of Medicago truncatula.

### 3. MtSHR in Cortical Cells is Involved in Nodule Symbiosis.

The inventors obtained the Tnt1 insertion mutant Mtshr2 (NF13823) of Medicago truncatula, and based on this plant material, the function of MtSHR1 was further inhibited. Specifically, MtSHR1-SRDX was fused downstream of the promoter of pK7WG2R-pMtNRT1.3, pK7WG2R-pMtSHR1 or pK7WG2R-pLjUBQ. The inventors constructed Medicago truncatula materials that differentially dominantly suppressed (including ubiquitous and root cortex-specific suppression) SHR1 gene function to some extent on the basis of SHR2 gene deletion, with the transgenic Medicago truncatula material containing an empty plasmid (EV) as a control.

The transgenic plants were planted in vermiculite and inoculated with Sm1021 at the 3rd day. After that, nodulation phenotype of the plants was observed at the 21st day after inoculation.

The results are shown in FIG. 6D, compared with the transgenic Medicago truncatula material expressing empty plasmid (EV), the number of nodules was significantly reduced in Medicago truncatula material that dominantly suppressed (pMtNRT1.3:SHR1-SRDX) SHR1 gene function to some extent on the basis of SHR2 gene deletion (FIG. 6D).

Both ubiquitous inhibition of SHR1 (pLjUBQ:SHR1-SRDX) and root cortex-specific inhibition of SHR1 (pMtNRT1.3:SHR1-SRDX) resulted in a significant reduction in the number of nodules, indicating that the SHR gene in the root cortex is involved in the formation of nodules and is vital for the formation and growth of nodules.

### Example 4. MtSHR-MtSCR Determines Cortical Cell Division of Medicago truncatula

1. MtSHR-MtSCR Determines Cortical Cell Division of Medicago truncatula after Rhizobia Treatment.

Root cortex cell division forms the nodule primordia. In this example, by analyzing the nodule primordia inoculated with rhizobia at a fixed point of the plant (under sterile conditions, tap the root with a toothpick dipped in bacterial liquid, and make a mark), the cortical cell division after rhizobia treatment was analyzed.

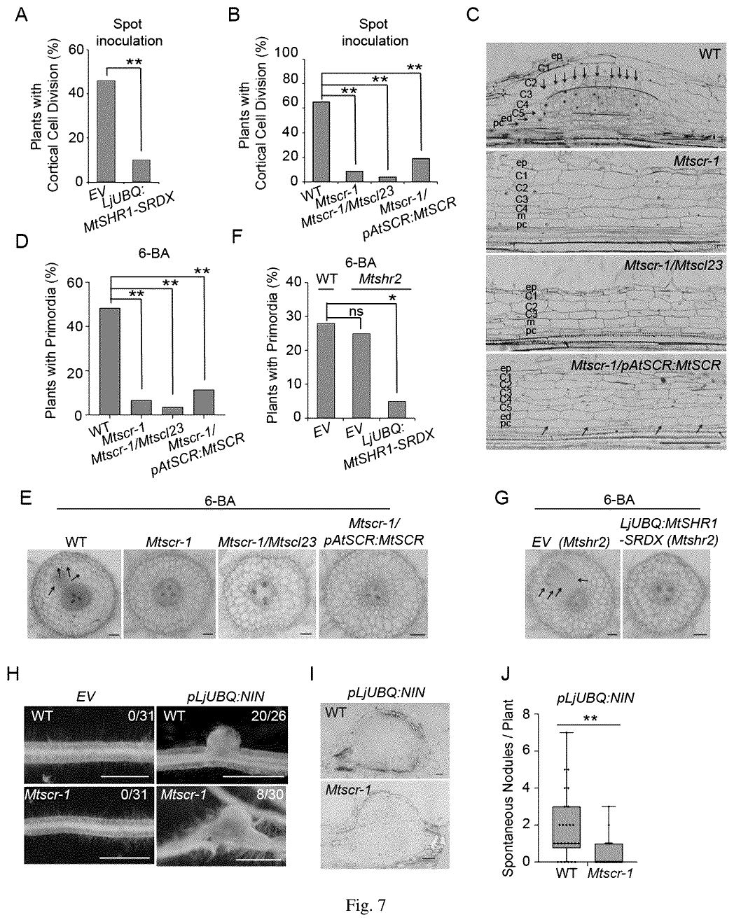

The inventors analyzed root cortical cell division of Medicago truncatula Tnt1 insertion mutant Mtscr-1, Mtscr-1/Mtscl23 double mutant, and analyzed pAtSCR:MtSCR stable transgenic plants with Mtscr-1 as the background. As shown in FIG. 7B-C, after spot inoculation with rhizobia, excessive root cortical cell division observed in the wild-type plants, while less root cortical cell division in the mutant Mtscr-1 and Mtscr-1/Mtscl23 double mutant, with root nodules formed abnormally. Compared with the wild-type plants, the root cortical cell division of the stable transgenic plants with pAtSCR:MtSCR was also significantly reduced, but recued to some extent. Therefore, cortically expressed SCR is essential for root cortical cell division in nodule formation.

At the same time, the inventors analyzed the nodule primordia of wild-type Medicago truncatula transformed with empty vector (EV) or pLjUBQ:MtSHR1-SRDX spot-inoculated with rhizobia in hairy root for 4 days. The results are shown in FIG. 7A, after rhizobia inoculation, EV transgenic plants showed a root cortical cell division, while the dominant suppressed plants expressing MtSHR1-SRDX driven by pLjUBQ promoters showed less root cortical cell division, due to MtSHR1 function inhibited by the inhibitory element SRDX.

Thus, the Medicago truncatula MtSHR-MtSCR determines the root cortical cell division response (to rhizobia).

2. MtSHR-MtSCR Determines Cortical Cell Division of Medicago truncatula after Cytokinins Treatment.

In the absence of rhizobia infection, the cortical cells of legumes can specifically respond to cytokinins and divide into pseudonodule. To further verify that MtSHR-MtSCR is a determinant factor controlling the division potential of cortical cells, the inventors treated wild-type, Mtscr-1, Mtscr-1/Mtscl23 and Mtscr-1/pAtSCR:MtSCR stable complementary plants with cytokinin (10 μM 6-BA) and counted the division of cortical cells by oscillating slice 4 days after treatment. The results showed that compared with the wild-type, ratios of cortical cell division in Mtscr-1, Mtscr-1/Mtscl23 and Mtscr-1/pAtSCR:MtSCR stable complementary plants were significantly reduced (FIG. 7D-E). At the same time, the inventors treated EV and pLjUBQ:MtSHR1-SRDX transformed hairy roots with 50 μM 6-BA respectively, and counted the cortical cell division of transgenic hair root by oscillating slice 4 days after treatment. The results showed that dominantly inhibited MtSHR1-SRDX significantly reduced the cortical cell division of hairy roots (FIG. 7F-G).

Thus, the Medicago truncatula MtSHR-MtSCR determines the root cortical cell division in response to cytokinin.

### 3. MtSCR is Involved in Spontaneous Nodulation Induced by NIN Overexpression.

NIN is a crucial transcription factor (Medtr5g099060) in the process of root nodule symbiosis. Without the infection of rhizobia, overexpression of NIN can trigger root cortical cell division to form nodule-like protrusions. In this example, the inventors overexpressed NIN in the wild type and Mtscr-1 respectively, and found that the number and proportion of spontaneous nodules produced by NIN overexpression in Mtscr-1 were significantly lower than those of the wild type (FIG. 7H-J), indicating that MtSCR is involved in the spontaneous nodulation caused by the overexpression of NIN.

### Example 5. Overexpressed SHR Induced Cortical Cell Division

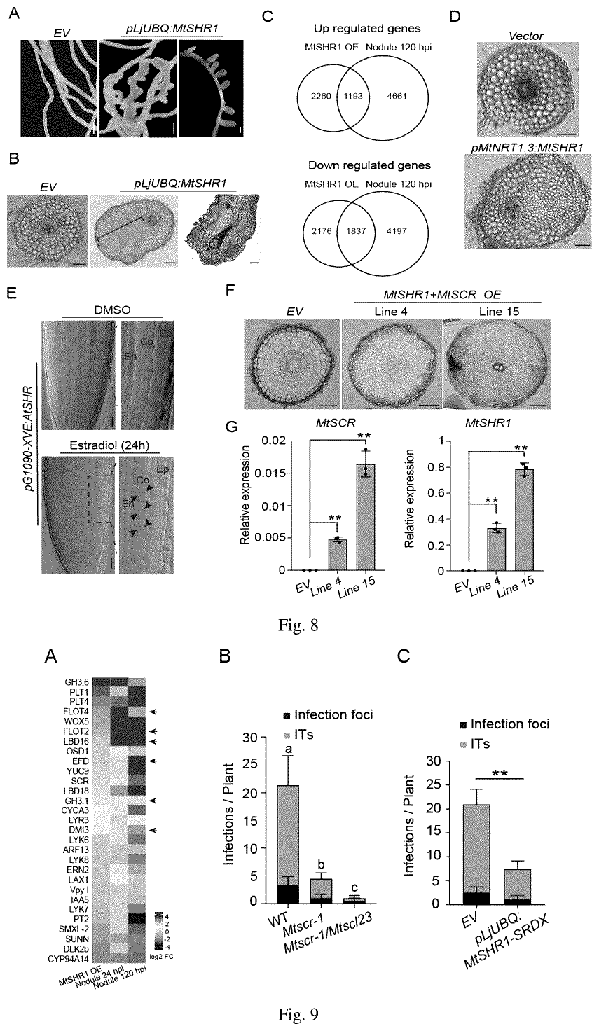

MtSHR-MtSCR governs the cortical cell division potential of Medicago truncatula. In order to investigate whether overexpression of MtSHR-MtSCR can induce cortical cell division of Medicago truncatula, the inventors overexpress MtSHR1 in Medicago truncatula hairy roots by the LjUBQ promoter (Hairy root transformation). pLjUBQ:MtSHR1 transgenic hairy roots have more cortical cells, with nodule-like pseudonodule formed in the absence of rhizobia inoculation (FIG. 8A-B).

The inventors further used EV and pLjUBQ:MtSHR1 transgenic hairy roots for RNA sequencing (RNAseq). Through enrichment analysis of differentially expressed genes, it was found that MtSHR1 overexpression caused changes in 7466 genes (1.5-fold; p<0.05). These differential genes were compared with the differential genes of the nodule tissue inoculated with rhizobia for 120 hours [the data of inoculated rhizobia are from (Schiessl et al., 2019)], and it was found that the proportion of genes that overlapped with the genes in the nodules inoculated with rhizobia for 120 hours was 40% (FIG. 8C), indicating that the cortical cell divisions induced by MtSHR1 overexpression were very similar at the transcriptome level to nodules inoculated with rhizobia for 120 hours, suggesting that these cell divisions showed part characteristics of nodules.

In addition, when the root cortical cell-specific promoter pMtNRT1.3 was used to drive MtSHR expression, cortical cell division was induced (FIG. 8D).

Finally, the inventors also found that the overexpression of SHR and SCR promotes cortical cell division, which is also conserved in non-legume Arabidopsis thaliana (FIG. 8E) and rice (FIG. 8F-G).

### Example 6. SHR-SCR Participates in Infection Thread Formation

In the sequencing results of SHR overexpression, the inventors noticed that the expression levels of some genes affecting the infection threads, such as FLOT4, FLOT2, EFD, GH3.1 and DM13, were all activated in the MtSHR1 overexpression material (FIG. 9A, black arrows), suggesting that MtSHR-MtSCR participates in infection thread formation. Consistent with this speculation, the inventors found that infection thread formation was severely defective in Mtscr-1, Mtscr-1/Mtscl23, and LjUBQ:SHR1-SRDX/Mtshr2 dominantly suppressed roots (FIG. 9B-C).

Combining the results of Example 5 and this example, it is indicated that MtSHR-MtSCR is involved in controlling both cortical cell division and infection thread formation.

### Example 7. SHR-SCR in Cortical Cells is Activated by Symbiotic Signals

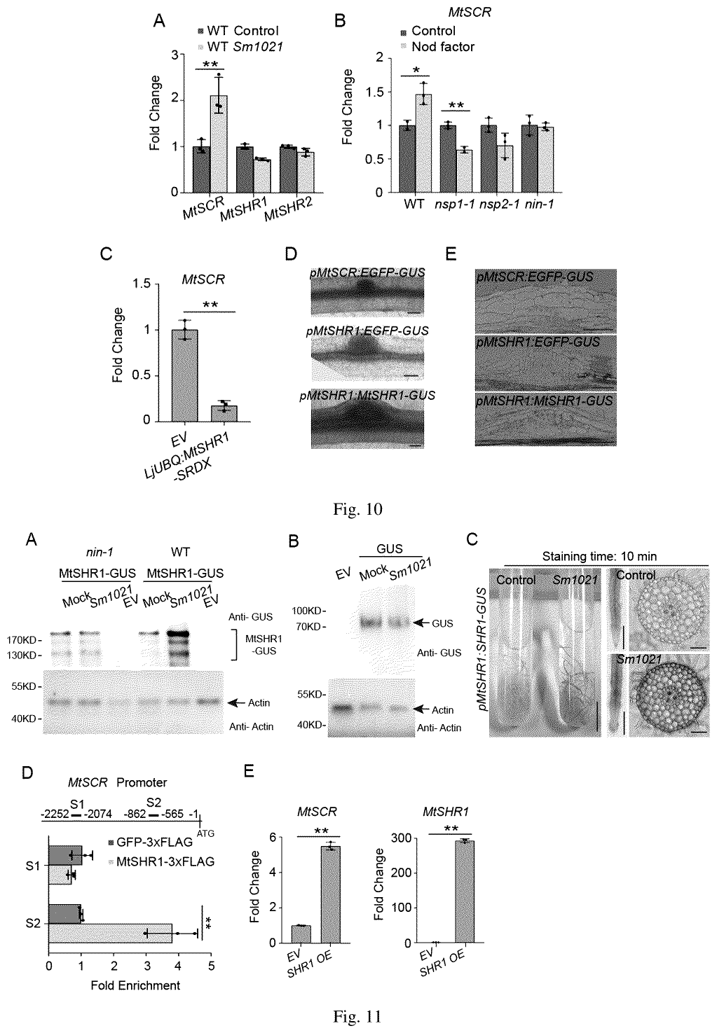

The inventors found that MtSCR expression was significantly increased in the wild-type roots inoculated with rhizobia (Sm1021) for 7 days (FIG. 10A). MtSCR expression was induced by Nod factor and was dependent on the symbiotic signaling components NSP1/NSP2/NIN (FIG. 10B). Overexpression of MtSHR activates MtSCR (FIG. 10D), consistent with the activation of AtSCR by AtSHR. In addition, the inventors also found that MtSCR expression in SHR-SRDX dominantly inhibited plants was significantly reduced (FIG. 10C), indicating that the induced expression of MtSCR also depends on MtSHR.

By GUS staining analyses of MtSCR and MtSHR1 promoters, the inventors found that MtSCR promoter is highly expressed in nodule primordia (FIG. 10D-E), while MtSHR1 promoter is only weakly or not expressed in nodule primordia (FIG. 10D-E). However, a strong GUS signal appeared in nodule primordia of pMtSHR1:MtSHR1-GUS transgenic hairy roots (FIG. 10D-E), suggesting that MtSHR1 moves into the cells of nodule primordia from cells of stele and cortex at nodule initiation.

The inventors further treated pMtSHR1:MtSHR1-GUS transgenic hairy roots with rhizobia, and proceed with GUS staining and Western blotting 3 days later. The results showed that rhizobia treatment promoted the accumulation of MtSHR1-GUS protein and depended on the symbiotic signal component NIN (FIG. 11A), but the inventors found that rhizobia treatment did not affect the GUS protein itself (FIG. 11B), indicating that the symbiotic signal altered the protein accumulation of MtSHR1. Sectioning results showed that the accumulated MtSHR1-GUS protein was mainly concentrated in cortical cells and epidermal cells (FIG. 11C).

The inventors also found that MtSHR1 can bind to the MtSCR promoter (FIG. 11D), wherein S1 is 2074 bp-2252 bp upstream of Medicago truncatula SCR promoter; S2 is 565 bp-862 bp upstream of Medicago truncatula SCR promoter. And, overexpression (OE) of MtSHR1 can activate the expression of MtSCR (FIG. 11E).

### DISCUSSION

Above all, through genetics, cell biology and molecular biology, the inventors found that spot inoculation of rhizobia can enrich MtSHR-MtSCR in cortical cells and nodule primordia (FIG. 10D-E, FIG. 11C). Overexpression of MtSHR-MtSCR in the cortex caused cortical cell division (FIG. 8D), with nodule-like structures formed without inoculation of rhizobia (FIG. 8A-B). Overexpression of MtSHR was able to induce the expression of genes associated with nodule development and infection thread formation (FIG. 9A), with 40% altered genes overlapping the transcriptome of 5-day-inoculated nodules (FIG. 8C).

Medicago truncatula MtSHR interacts with MtSCR (FIG. 5). Although MtSHR, like AtSHR, is specifically expressed only in the stele of roots (FIG. 6A), MtSHR protein can move to cortical cells and epidermal cells of Medicago truncatula (FIG. 6B) By cortex-specifically interfering the function of MtSHR, it was demonstrated that cortically accumulated MtSHR controls early cortical cell division in nodule formation (FIG. 6D). By rhizobia spot inoculation and cytokinin treatments, it is demonstrated that the MtSHR-MtSCR of cortical cells determines the cortical cell division potential of Medicago truncatula (FIG. 7).

Activation of the MtSHR-MtSCR module in non-legumes Arabidopsis thaliana and rice also induced root cortical cell division (FIG. 8E-G). FIG. 8E shows that induction of MtSHR expression in Arabidopsis thaliana cortical cells induces root cortical cell division. FIGS. 8F-G show that overexpression of MtSHR-MtSCR in rice can also induce root cortical cell division. The above data indicated that the MtSHR-MtSCR module is a sufficient and necessary condition for the activation and division of root cortical cells, suggesting that the ectopic expression of MtSHR-MtSCR in cortical cells is a decisive factor for the root cortical cell division potential of legumes.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1. A method for identifying plant traits, comprising: analyzing promoter of plants' gene SCARECROW; if cis-elements AT1 Box and Enhancer are both present, then the gene SCARECROW is expressed normally, and the plant traits are normal; if either one of AT1 Box and Enhancer is deleted, then gene expression of SCARECROW is abnormal, and the traits are abnormal;
wherein, the traits comprise: formation of infection threads, ability of cortical cells to respond to cytokinins, ability of cortical cells to respond to rhizobial infection, NIN-mediated plant spontaneous nodulation, cortical cell division or nodule organogenesis.

2. A method for selecting plants with normal traits, comprising: analyzing the promoter of plants' gene SCARECROW; wherein, if cis-elements AT1 Box and Enhancer are both present, it indicates that the gene SCARECROW is expressed normally and the plant traits are normal; wherein, the traits comprise formation of infection threads, ability of cortical cells to respond to cytokinins and rhizobial infection, NIN-mediated plant spontaneous nodulation, cortical cell division or nodule organogenesis.

3. (canceled)

4. The method according to claim 1, wherein the plants with normal traits are plants that form root nodule tissues or root nodule-like tissues.

5. The method according to claim 1, wherein the cis-element AT1 Box has a nucleotide sequence shown in SEQ ID NO: 28 or a nucleotide sequence with more than 80% sequence identity to said nucleotide sequence; and/or
the sequence of the cis-element Enhancer is GANTTNC, wherein the N represents A, T, C or G.

6. The method according to claim 1, wherein the plants are selected from the following groups comprising:
plants expressing gene SCARECROW;
rhizobial plants;
Gramineous; and/or
Brassicaceae.

7. A method for improving traits of Leguminosae or Gramineous plants, wherein it comprises increasing the expressions or activities of SCARECROW and SHORTROOT in plants, or promoting the interaction of SCARECROW and SHORT ROOT;
wherein, the improved traits are selected from the following group comprising: promoting the formation of infection threads, changing identity of cortical cells, improving abilities of cortical cells to respond to cytokinins, improving ability of cortical cells to respond to rhizobial infection, promoting NIN-mediated plant spontaneous nodulation, promoting cortical cell division, promoting nodule organogenesis.

8. The method according to claim 7, wherein SCARECROW and/or SHORT ROOT are ectopically expressed in the cortex.

9. The method of claim 8, wherein the expression is performed using a cortical cell-specific promoter or a ubiquitous promoter.

10. The method of claim 9, wherein the cortical cell-specific promoter comprises: NRT1.3 promoter; or
the ubiquitous promoter comprises: LjUBQ promoter.

11. The method according to claim 7, wherein the promotion of interaction of SCARECROW and SHORT ROOT in plants is to promote the combination of SHORT ROOT with the promoter of gene SCARECROW.

12. The method according to claim 7, wherein the elevation of expression or activity of SCARECROW and SHORT ROOT in plants, or the promotion of the interaction of SCARECROW and SHORT ROOT comprises:
Transforming genes SCARECROW and SHORT ROOT or expression constructs or vectors containing said gene into plants;
Improving expressive efficiency of genes SCARECROW and SHORT ROOT in plants by enhancers or tissue-specific promoters;
Increasing expressive efficiency of genes SCARECROW and SHORT ROOT in plants by enhancer; or
Exogenously adding the cis-element AT1 Box or Enhancer of gene SCARECROW in the promoter when the element is absent in plants.

13. (canceled)

14. A method for screening substances for improving traits of Leguminosae or Gramineous plants, wherein the method comprises:
(1) Adding candidate substance to the system containing protein SHORT ROOT and gene SCARECROW, wherein gene expression of the SCARECROW is driven by its promoter;
(2) Detecting the system to observe the interaction between SHORT ROOT and promoter of SCARECROW in the system of (1); if the candidate substance promotes the combination of two, or promotes the expression of pSCR in cortical cells, then the candidate substance is the substance improving traits of legumes or gramineous plants;
wherein the improved traits are selected from the following group comprising: promoting the formation of infection threads, changing identity of cortical cells, improving abilities of cortical cells to respond to cytokinins, improving abilities of cortical cells to respond to rhizobial infection, promoting NIN-mediated plant spontaneous nodulation, promoting cortical cell division, promoting nodule organogenesis.

15. The method according to claim 1, wherein the cortical cells comprise: root cortical cells or epidermal cells.

16. The method according to claim 1, wherein the cis-element AT1 Box has a nucleotide sequence selected from any one of SEQ ID NO: 15-24; or, the sequence of the cis-element Enhancer has a nucleotide sequence shown in any one of SEQ ID NO: 5-14.

17. The method according to claim 6, wherein the rhizobial plants comprise Leguminosae; or, the Gramineous comprise: rice, barley, wheat, oats, rye, corn, sorghum.

18. The method according to claim 17, wherein the Leguminosae comprise Medicago truncatula, Glycine, Lotus, Pisum, Cicer, Lupinus, Phaseolus, Trifolium and Parasponia.
